(19) United States
(12) Patent Application Publication
(10) Pub. No.: US 2018/0177729 A1
(43) Pub. Date: Jun. 28, 2018

(54) G PROTEIN-COUPLED RECEPTORS INCORPORATED INTO REHYDRATED POLYMER VESICLES RETAIN FUNCTIONALITY

(71) Applicant: UNIVERSITY OF SOUTHERN CALIFORNIA, LOS ANGELES, CA (US)

(72) Inventors: MARY G. GUTIERREZ, SAN BRUNO, CA (US); NOAH MALMSTADT, LOS ANGELES, CA (US); CARSON RICHE, ZIONSVILLE, PA (US)

(21) Appl. No.: 15/855,353

(22) Filed: Dec. 27, 2017

Related U.S. Application Data
(60) Provisional application No. 62/439,174, filed on Dec. 27, 2016.

(51) Int. Cl. A61K 9/127; A61K 47/42

(52) U.S. Cl. CPC A61K 9/1273; A61K 47/42

(57) ABSTRACT

A G Protein Coupled Receptor (GPCR) is incorporated in polymeric giant unilamellar protein vesicles (pGUPs). By utilizing an agarose rehydration technique, the GPCR is inserted in the biased, physiological orientation with the C-terminus cytosolic and N-terminus extracellular. The GPCR is fully functional within the polymeric bilayer, exhibiting physiological responses to various ligands. The entire population of GPCRs in pGUPs remains fully functional after lyophilization for 120 hours.

### CROSS-REFERENCE TO RELATED APPLICATIONS

This application claims the benefit of U.S. provisional application Ser. No. 62/439,174 filed Dec. 27, 2016, the disclosure of which is hereby incorporated in its entirety by reference herein.

### STATEMENT REGARDING FEDERALLY SPONSORED RESEARCH OR DEVELOPMENT

The invention was made with Government support under Contract No. R01 GM093279 awarded by the National Institute of Health, and under Contract No. N00014-12-1-0620 awarded by the Office of Naval Research. The Government has certain rights to the invention.

### TECHNICAL FIELD

In at least one aspect, the present invention is related to polymersomes formed from polymeric membranes.

### BACKGROUND

Since their discovery in 1999 polymersomes have been used as biomimetic platforms to better understand physiological and material properties of cells.¹ Compared to their liposomal counterparts, polymersomes display greater stability and decreased permeability²⁻³ and therefore have been exploited and hybridized with cellular components such as lipids and proteins for drug delivery and research.⁴ For example, phase separation has been modeled in lipid-polymer vesicles offering a platform for “windows” of lipid bilayers to be observed in a polymeric framework.⁵ The encapsulation of nanometer sized polymersomes within giant unilamellar vesicles (GUVs) further provides insight into the compartmentalization of living cells.⁶ In recent years protein incorporation into varying lamellar phase polymeric vesicles has been reported.⁷⁻⁸

G protein-coupled receptors (GPCRs) are a class of proteins targeted for membrane vesicle incorporation.⁹ As druggable targets, there exists over 800 GPCRs with almost half of the therapeutics on the market targeting these proteins.¹⁰ GPCRs are characterized by seven transmembrane alpha helices; they are integral membrane protein receptors associated with cytoplasmic G proteins consisting of α, β, and γ subunits. Binding of an extracellular agonist to the receptor causes a conformational change and dissociation of the G subunits into Gα and βγ. Exchange of GDP for GTP on the Gα subunit results in intracellular signal cascades responsible for many cellular processes such as apoptosis, proliferation and changes in intracellular cyclic adenosine monophosphate (cAMP) levels.^{9, 11}

Incorporation of GPCRs into polymersomes via cell free expression has been previously demonstrated. In 2014, May et al. incorporate the dopamine receptor D₂ (DRD2) into polymeric vesicles.¹² However, they only observed ligand binding on nanoscale vesicles and were not able to show functionality since the G protein subunits were not present. Using the same approach de Hoog et al. incorporated the chemokine C-X-C motif receptor 4 (CXCR4) into polymersomes and tracked its binding to antibodies via surface plasmon resonance.¹³ While these approaches incorporate GPCRs into polymersomes they are limited by 1) the need for encapsulation of expression components, 2) the lack of cognizant G protein subunits, and 3) liposomal sized vesicles of 100-150 nm make them inaccessible to microscopy.

Accordingly, there is a need for improved polymersomes in which fully functional GPCRs can be incorporated.

### SUMMARY

The present invention solves one or more problems of the prior art by providing in at least one embodiment a composition comprising a unilamellar polymersome. The polymersome includes an exterior surface and an interior surface with the interior surface defining a central cavity. The unilamellar polymersome includes a polymeric layer and at least one functional G protein-coupled receptor incorporated into the polymeric bilayer. In a refinement, the polymeric layer is a polymeric bilayer. In a further refinement, polymer layer includes a block copolymer that includes a hydrophobic block and a polymer block having a polar group such as oxygen.

### BRIEF DESCRIPTION OF THE DRAWINGS

FIGS. 1A, 1B, and 1C. GPCR incorporation into polymeric vesicles, pGUPs. (A) Schematic of pGUP formation and protein incorporation. Films of protein, agarose, and polymer are made on a coverslip and rehydrated with a sucrose buffer solution containing BODIPY-GTPγS. pGUPs can be lyophilized and the GPCR retains its function (steps 2-4). (B) Confocal micrographs of pGUPs prior to lyophilization. The left micrograph shows the polymer bilayer tagged with ATTO-488-DPPE. The right micrograph shows that rhodamine antibody-tagged 5-HT_{1A}R is evenly distributed throughout the polymer bilayer. (C) The left image shows a pGUP sample after lyophilization. Upon rehydration, pGUPs can still be detected as shown in the right micrograph. All scale bars represent 5 μm.

FIG. 2. 5-HT_{1A}R agonist activated functionality as determined by BODIPY-GTPγS binding to G proteins. Fluorescence unquenching due to the irreversible binding of BODIPY-GTPγS to G proteins was tracked for 12 hours. 5-HT_{1A}R incorporated into pGUPs exhibit protein function. Decreasing the amount of antagonist spiperone in the system increases the protein functional rate (See Table 1 for best-fit rates). Control curves shown in the in-set are samples of indicated concentration incubated without agonist and indicate protein basal activity levels.

FIG. 3. Functional rates of 5-HT_{1A}R in polymersomes (pGUPs) and diluted in various solutions. Controls (Ctl) are plotted alongside agonist-exposed samples (+Ag). The percent intensity increase of the samples indicates the population of functional protein. In Milli-Q water and PBS, the 5-HT_{1A}R displays no fluorescence activity. In 200 mM sucrose in PBS (pH 7.4) 5-HT_{1A}R displays weaker fluorescence intensity increase compared to pGUPs. Furthermore, there is no difference in rate between the Ctl and +Ag protein in 200 mM sucrose in PBS.

FIG. 4. Retained fluorescence intensity of quenched monoclonal antibody-tagged pGUPs. pGUPs were formed with monoclonal rhodamine antibody-tagged receptor or G protein and subsequently quenched. Retained fluorescence intensity indicates the population of receptor in the correct orientation and G Protein in the inner leaflet of the vesicles. Over 90% of the receptor population is incorporated in the correct orientation while G proteins exists across both leaflets. Control pGUPs were made with ATTO-488-DPPE throughout both bilayers; this lipid tag is quenched ˜50%.

FIG. 5. pGUP formation using the agarose swelling method for protein incorporation. The micrographs depicted are DIC time-lapse images as indicated above each image in seconds. Time 0 s represents the hydrogel and polymer film immediately prior to rehydration with 200 mM sucrose in PBS (pH 7.4). Vesicle formation begins almost immediately and pGUPs start as nanometer size vesicles and continue to grow and form micrometer sized vesicles. Coalescence of the smaller vesicles into large vesicles is captured in the decrease in number of vesicles from 60 s to 180 s to 300 s. The plot below the images shows the pGUP count and average radius in μm over a 5-minute period. pGUP count initially spikes and decreases while radius increases and plateaus over time, indicating the coalescence of vesicles during pGUP formation.

FIGS. 6A, 6B, and 6C. Comparison of pGUPs before and after lyophilization. A) The left micrograph shows a typical yield of pGUPs on the hydrogel before harvesting. The right micrograph shows a patch of pGUPs that were lyophilized on the hydrogel for viewing purposes. pGUPs are smaller and display a rough perimeter due to dehydration from lyophilization. B) This set of images shows pGUPs after they have been harvested from the hydrogel and settled. The left micrograph shows pGUP controls that were not lyophilized. The right micrograph shows typical pGUPs after lyophilization and subsequent rehydration with Milli Q water. C) Histograms of size distributions of pGUPs before lyophilization and after lyophilization. Prior to lyophilization the mean average radius of a typical pGUP population is 6.03±0.24 After lyophilization the mean average radius of a typical pGUP population is 5.94±0.24

FIG. 7. Results of pGUPs without spiperone. pGUPs were formed without the antagonist, spiperone, in the rehydration buffer and were lyophilized for 0 hour (0 h, non-lyophilized), 24 hours or 120 hours then assayed for protein functionality. For all three time periods, control and the 24 h and 120 h lyophilization and rehydration, the protein continues to display inherent functionality and agonist induced activity, however, the rate of protein function for the lyophilized samples are attenuated compared to the 0 h non-lyophilized control. (See Table 3 for quantitative rates). The control curves in the in-set are pGUP samples of the indicated lyophilization time that were not incubated with agonist, and therefore indicate protein basal activity.

FIG. 8. Normalized rate of agonist and control protein diluted in 200 mM sucrose in PBS. As presented in FIG. 4 of the main text, 5-HT_{1A}R membrane fragments diluted in sucrose rehydration buffer shows protein function; however, they do not show agonist dependence as protein exposure to 8-OH-DPAT does not show an increase in protein functional rate. The curves are averages of the data points presented. The rates as determined by a single exponential filling of the control protein is 7.23±0.12 (×10⁻² min⁻¹) and the rate of the agonist-incubated protein is 7.18±0.12 (×10⁻² min⁻¹).

FIG. 9. Curves of protein functional rate with varying antagonist. The points indicate the average of 6 pGUP observations for each of the listed antagonist, methiothepine maleate, NAN-190, and WAY 100635, and the shaded area around the points are the standard error mean. The inset shows the control pGUPs that were not incubated with agonist and represents protein basal activity in the specified pGUP samples.

### DETAILED DESCRIPTION

Reference will now be made in detail to presently preferred compositions, embodiments and methods of the present invention, which constitute the best modes of practicing the invention presently known to the inventors. The Figures are not necessarily to scale. However, it is to be understood that the disclosed embodiments are merely exemplary of the invention that may be embodied in various and alternative forms. Therefore, specific details disclosed herein are not to be interpreted as limiting, but merely as a representative basis for any aspect of the invention and/or as a representative basis for teaching one skilled in the art to variously employ the present invention.

Except in the examples, or where otherwise expressly indicated, all numerical quantities in this description indicating amounts of material or conditions of reaction and/or use are to be understood as modified by the word “about” in describing the broadest scope of the invention.

Practice within the numerical limits stated is generally preferred. Also, unless expressly stated to the contrary: percent, “parts of,” and ratio values are by weight; the term “polymer” includes “oligomer,” “copolymer,” “terpolymer,” “block”, “random,” “segmented block,” and the like; the description of a group or class of materials as suitable or preferred for a given purpose in connection with the invention implies that mixtures of any two or more of the members of the group or class are equally suitable or preferred; description of constituents in chemical terms refers to the constituents at the time of addition to any combination specified in the description, and does not necessarily preclude chemical interactions among the constituents of a mixture once mixed; the first definition of an acronym or other abbreviation applies to all subsequent uses herein of the same abbreviation and applies mutatis mutandis to normal grammatical variations of the initially defined abbreviation; and, unless expressly stated to the contrary, measurement of a property is determined by the same technique as previously or later referenced for the same property.

It is also to be understood that this invention is not limited to the specific embodiments and methods described below, as specific components and/or conditions may, of course, vary. Furthermore, the terminology used herein is used only for the purpose of describing particular embodiments of the present invention and is not intended to be limiting in any way.

It must also be noted that, as used in the specification and the appended claims, the singular form “a,” “an,” and “the” comprise plural referents unless the context clearly indicates otherwise. For example, reference to a component in the singular is intended to comprise a plurality of components.

The term “comprising” is synonymous with “including,” “having,” “containing,” or “characterized by.” These terms are inclusive and open-ended and do not exclude additional, unrecited elements or method steps.

The phrase “consisting of” excludes any element, step, or ingredient not specified in the claim. When this phrase appears in a clause of the body of a claim, rather than immediately following the preamble, it limits only the element set forth in that clause; other elements are not excluded from the claim as a whole.

The phrase “consisting essentially of” limits the scope of a claim to the specified materials or steps, plus those that do not materially affect the basic and novel characteristic(s) of the claimed subject matter.

With respect to the terms “comprising,” “consisting of,” and “consisting essentially of,” where one of these three terms is used herein, the presently disclosed and claimed subject matter can include the use of either of the other two terms.

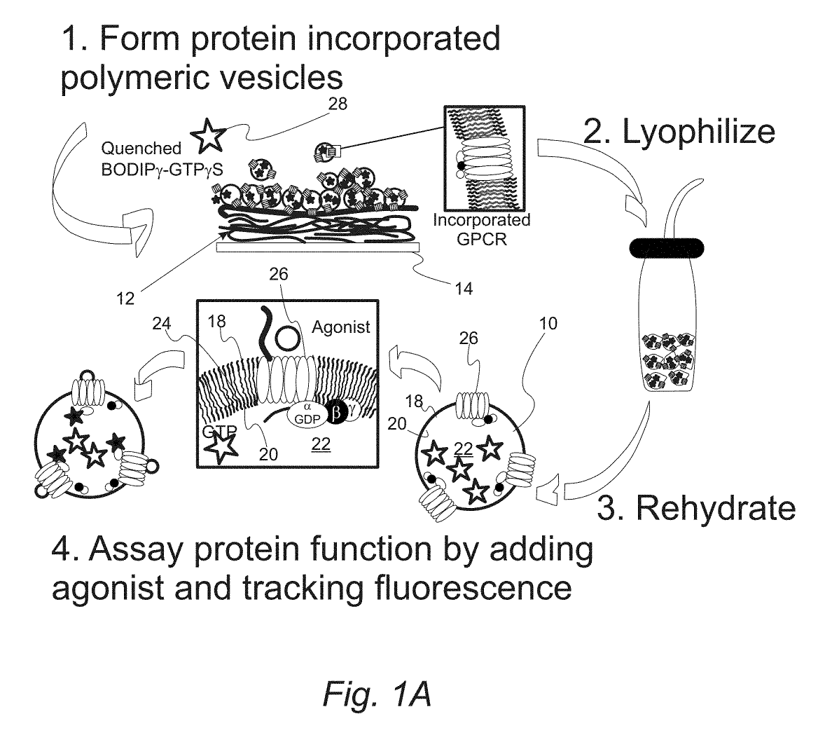

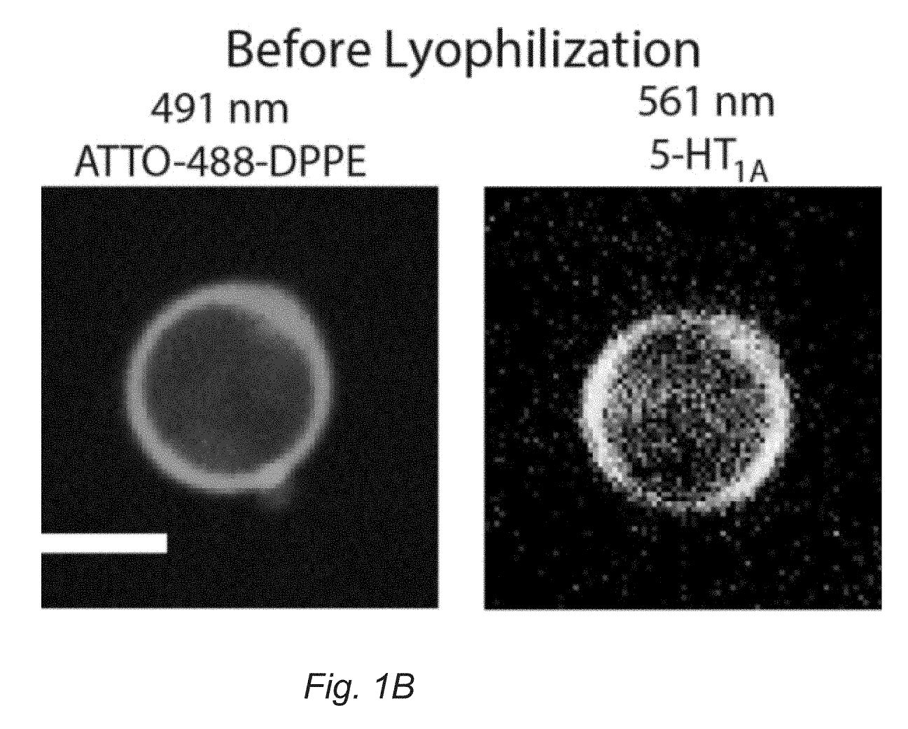

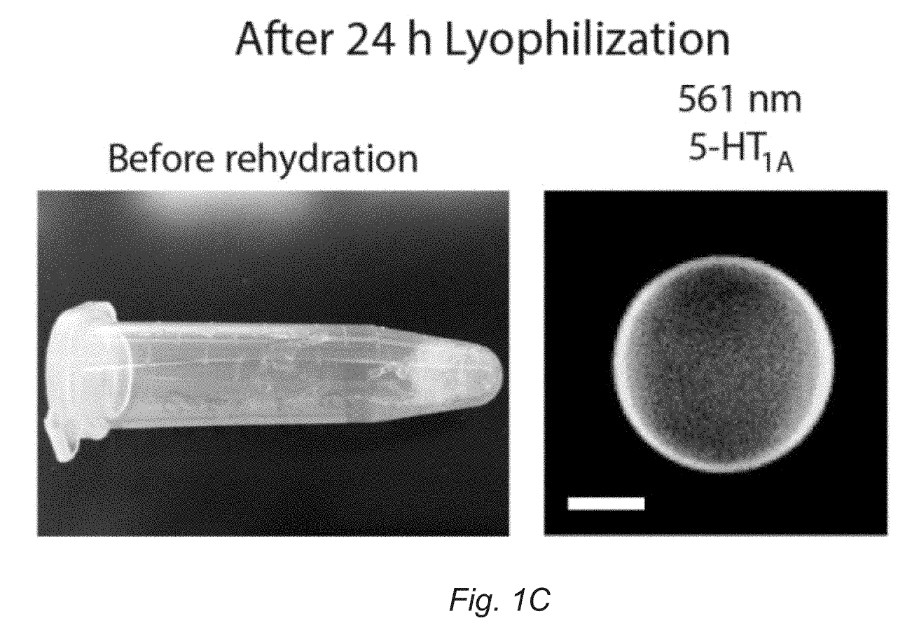

With reference to FIG. 1A, a schematic showing the formation of a unilamellar polymersome in accordance to an embodiment of the present invention is provided. Polymersome 10 is formed by forming a film 12 of a protein (e.g., G protein-coupled receptor), polymer, and an optional payload compound on a substrate 14 (e.g., a coverslip). The film is rehydrated (e.g., with a sugar-containing solution) to form polymersome 10. Polymersome 10 includes an exterior surface 18 and an interior surface 20 with the interior surface defining a central cavity 22. The unilamellar polymersome includes a polymeric layer 24 and at least one functional G protein-coupled receptor 26 incorporated into the polymeric layer 24. In a refinement, the polymeric layer is a polymeric bilayer. In a refinement, a payload compound 28 is disposed within the central cavity. Such payload molecules can be a fluorescent compound, a therapeutic compound, and the like.

In a further refinement, polymer layer includes a block copolymer that includes a hydrophobic block and a block having a polar group (e.g., a hydrophilic polymer block). Oxygen is an example of such a polar group. The polymersomes generally comprise block copolymers that comprise a hydrophobic block and a hydrophilic block. These block copolymers may have a number average molecular weight in the range of from about 2,000 to about 100,000. In an exemplary embodiment of the present invention, the block copolymers may have a hydrophilic mass fraction in the range of from about 20% to about 45% by weight of the block copolymer. In an exemplary embodiment of the present invention, the block copolymers may have a hydrophobic mass fraction in the range of from about 55% to about 80% by weight of the block copolymer.

In a variation, the hydrophilic block includes polymers that are soluble in water. Examples of suitable polymers for the hydrophilic block include, but are not limited to, poly(ethylene glycol), poly(2-methyloxazoline), poly(acrylic acid), poly(ethylene oxide), poly(methacrylic acid), poly(2-acrylamido, 2-methyl propane sulfonic acid), poly(acrylamide), poly(2-dimethylaminoethyl methacrylate), and derivatives thereof, and combinations thereof. In contrast, the hydrophobic block includes a polymer which is not soluble in water. Examples of suitable hydrophobic polymers for the hydrophobic block include, but are not limited to, polydimethyl siloxane, poly(caprolactone), poly(lactide), poly(methylmethacrylate), poly(propylene), poly(butadiene), poly(styrene), poly(isoprene), poly(ethylene), poly(ethylene propylene), poly(ethylene butene), and derivatives thereof, and combinations thereof. Particularly useful examples for the polymer layer having hydrophobic and hydrophilic block are poly(butadiene-b-ethyleneoxide) and poly(butadiene-b-acrylic acid).

Characteristically, due to the structural similarities between the different GPCR families, the present invention is not limited by the type of GPCR that is incorporated into the polymersome membrane. In particular, the GPCRs that are integrated into the polymersome membranes can be belong to the following receptor classes: rhodopsin-like receptor, receptors of the secretin receptor family, metabotropic glutamate/pheromone receptors, fungal mating pheromone receptors, cyclic AMP receptors, and frizzled/smoothened). Specific examples of GPCRs include, but are not limited to, angiotensin receptors, bombesin receptors, bradykinin receptors, calcitonin, parathyroid hormone, secretin receptors, chemokine receptors, chemotactic peptide receptors, c5a receptor, cholecystokinin/gastrin receptors, corticotropin (ACTH) receptors, endothelin receptors, glycoprotein hormones receptors, melanocortin receptors, motilin receptors, neuropeptide Y receptors, neurotensin receptors, oploid receptors, mu opioid receptor, delta opioid receptor, kappa opioid receptor, nociceptin, opioid receptor, releasing hormone receptors, somatostatin receptors, tachykinin receptors, thrombin/protease receptors, vasopressin/oxytocin receptors, acetylcholine (muscarinic) receptors, adrenergic receptors, dopamine receptors, histamine receptors, serotonin receptors, adenosine and other adenine nucleotide receptors, cannabinoids receptors, prostanoids and paf receptors, calcium receptors, opsins, viral receptors, and orphan receptors.

The following examples illustrate the various embodiments of the present invention. Those skilled in the art will recognize many variations that are within the spirit of the present invention and scope of the claims.

To overcome the limitations of the prior art, the present invention provides a robust platform for incorporation of GPCRs into giant unilamellar polymersomes in the micrometer range that allows for observations of GTP/GDP exchange on G proteins catalyzed by the human serotonin 5-HT_{1A} receptor (5-HT_{1A}R). In at least one embodiment, the present invention exploits the stability of polymersomes and shows that following lyophilization and rehydration, polymeric giant unilamellar protein-vesicles (pGUPs) with integrated GPCRs retain vesicle integrity and protein function. The agarose hydration method previously reported is utilized for the incorporation of 5-HT_{1A}R into giant unilamellar vesicles.¹⁴⁻¹⁵ Preparations of 5-HT_{1A}R are incorporated with associated G proteins into polymeric membranes made of polybutadiene-b-poly(ethylene oxide) (PBd(650)-PEO(400)). To detect the functionality of 5-HT_{1A}R, pGUPs were formed to encapsulate BODIPY-GTPγS, a quenched fluorophore. When an agonist binds to 5-HT_{1A}R on the pGUPs, G protein subunits exchange bound GDP for BODIPY-GTPγS and this exchange unquenches its fluorescence (FIG. 1). Using this system, we detect physiological responses of 5-HT_{1A}R in the presence of different antagonists and further show retained protein function after lyophilizing and rehydrating pGUPs.

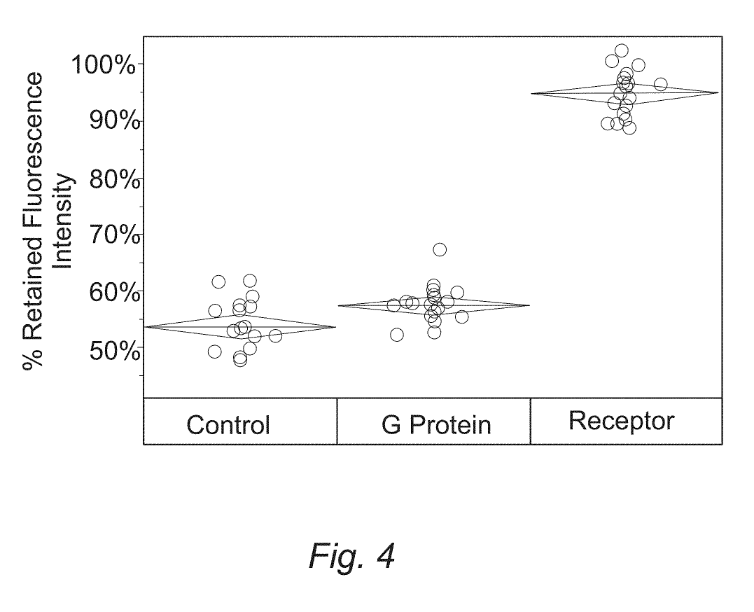

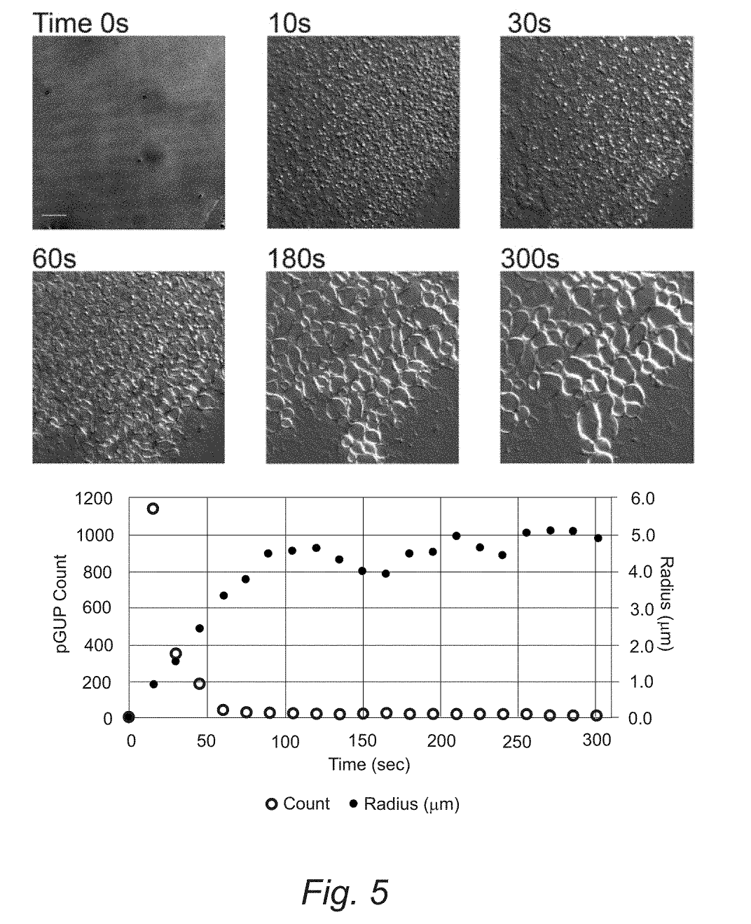

FIG. 1B shows the successful incorporation of 5-HT_{1A}R membrane preparations into polymer bilayer membranes. 5-HT_{1A}R tagged with rhodamine-labeled antibodies is evenly distributed throughout the bilayer (FIG. 1B, right panel). To determine if 5-HT_{1A}R is correctly oriented in pGUPs, monoclonal rhodamine-antibody tagged 5-HT_{1A}R or G proteins were incorporated into pGUPs and subsequently quenched with the membrane impermeable fluorescence-quenching agent QSY7. The monoclonal 5-HT_{1A}R antibody is known to bind to the cytosolic face of the receptor, thus any quenched fluorophore would indicate incorporated receptors with incorrect orientation. Results of retained fluorescence after quenching are presented in FIG. 4. Less than 10% of the receptor-labelled antibody fluorescence is quenched, indicating that 5-HT_{1A}R displays a biased orientation with the C-terminus cytosolic and the N-terminus extracellular. The peripheral G protein subunits are distributed in both polymer bilayer leaflets without a bias towards either the inner or outer leaflet; only ˜55% of the G protein-labeling antibody fluorescence is retained after quenching. The results indicate the formation of pGUPs using the agarose technique is likely the initial formation of nanoscale liposomes; the high curvature causes proteins to orient themselves within the bilayer.¹⁷⁻¹⁸ These liposomes then coalesce into larger pGUPs during the agarose rehydration process to form giant vesicles on the micrometer scale (See FIG. 5).

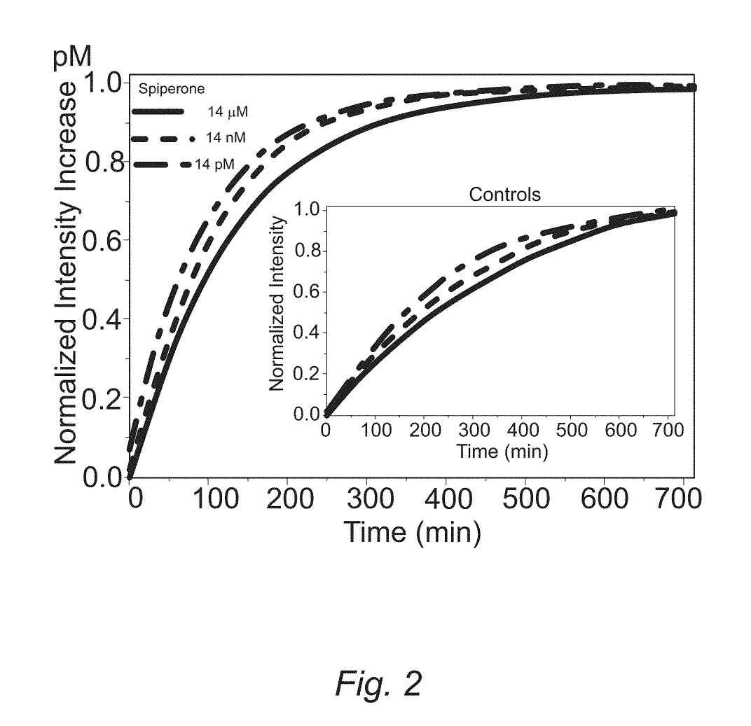

To observe protein function in the synthetic polymer bilayers, pGUPs were formed, settled in glucose and transferred to a 96-microtiter plate. pGUPs were formed in the presence of antagonist, spiperone (final concentration 14 μM unless otherwise stated), to reduce protein basal activity and incubated with agonist 8-Hydroxy-2-(dipropylamino)tetralin hydrobromide (8-OH-DPAT) at 37° C. for 12 hours. Fluorescence unquenching of BODIPY-GTPγS due to G protein binding was monitored every 5 minutes. Tracked fluorescence over time was first calculated as percent intensity increase to account for variation in sample size. Curves represent six independent replicates of the same experimental protocol and each time point is an average of the intensity increase. Curves were then normalized for ease in viewing the differences in protein functional rate. As shown in FIG. 2, agonist activated fluorescence intensity of the pGUPs increases over time indicating that 5-HT_{1A}R functions and is agonist induced in the polymeric membranes.

5-HT_{1A}R in pGUPs displays physiological responses to varying amounts of antagonist. Reducing the final concentration of spiperone in pGUPs from 14 μM to 14 results in an expected increase in the protein functional rate (FIG. 2). Furthermore, other known 5-HT_{1A}R antagonist, methiothepin maleate (methiothepin), NAN-190, and WAY 100635, reduce the protein functional rate. The reported Ki values for spiperone and methiothepin ranges between 7-8 nM,¹⁹ while NAN-190 and WAY 100635 are reported to be 0.55 nM and 0.84 nM respectively.²⁰⁻²¹ The reported values vary depending on the cell lines that are used,²¹ nevertheless the observed trends in the rates reported in Table 1 are consistent with the expected physiological response of 5-HT_{1A}R.

TABLE 1
Physiological 5-HT_{1A}R in pGUP response to changes in antagonist
species and concentration.

| | Rate | Std Error |
|---|---|---|
| Antagonist | (×10⁻² min⁻¹) | (×10⁻² min⁻¹) |
| 14 pM Spiperone | 10.2 | 0.3 |
| 14 nM Spiperone | 9.6 | 0.3 |
| 14 μM Spiperone | 7.7 | 0.1 |
| 14 μM Methiothepin | 8.5 | 0.2 |
| 14 μM NAN-190 | 5.7 | 0.1 |
| 14 μM WAY 100635 | 5.9 | 0.1 |

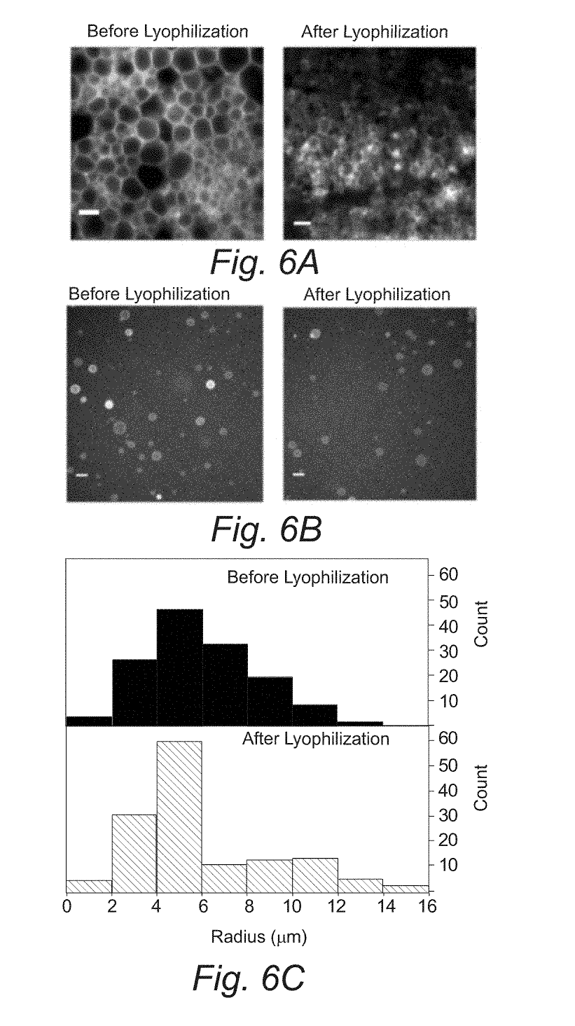

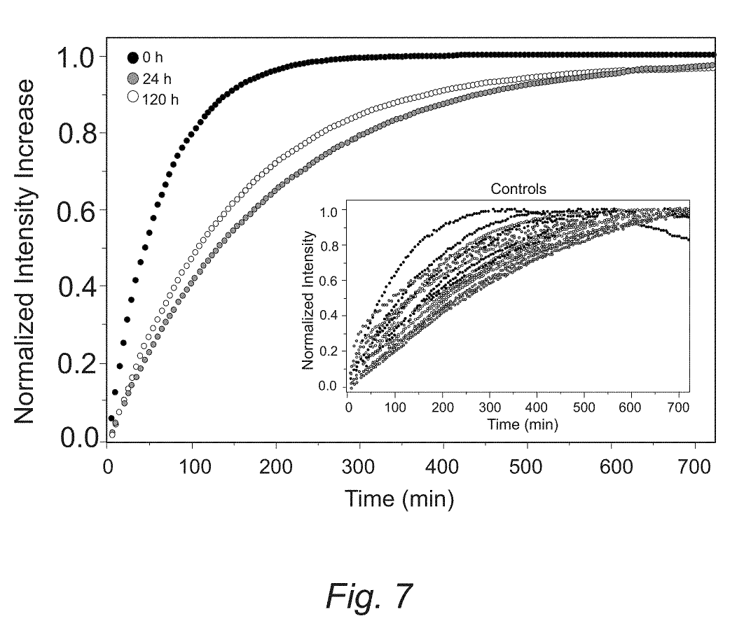

A remarkable feature of the 5-HT_{1A}R pGUPs is their stability over cycles of lyophilization and rehydration. Freeze drying of proteins often renders them nonfunctional and larger lipid vesicles (>5 μm) typically display fracturing upon lyophilization.²²⁻²³ Since polymersomes are known for increased stability we formed pGUPs as described above and subjected them to flash freezing for five minutes in liquid nitrogen followed by overnight vacuum at 0.5 torr and −35° C. to completely lyophilize the samples. pGUPs were kept frozen with desiccant at −20° C. for extended storage. At 24 hours and 120 hours, lyophilized samples were rehydrated with deionized water (37° C.) for 20 minutes. Rehydrated samples were observed using epifluorescence microscopy and analyzed via fluorescence microtiter plate assay as previously described. 24 hours and 120 hours pGUPs were still vesicular and retained their size (FIG. 6). Furthermore, lyophilized pGUPs display protein functional rates comparable to that of control pGUPs that were not lyophilized (Table 2). A Tukey-Kramer pair wise comparison of means (α=0.05) shows that the differences in rates are not statistically significant. The percent intensity increases of the pGUPs accounts for varying amounts of pGUPs in individual microtiter wells and is also indicative of the population of functional receptors. Table 2 shows that the percent intensity increase does not vary significantly across all samples and thus the functional protein populations in pGUPs do not decrease upon lyophilization. pGUPs formed without spiperone and subjected to lyophilization, also displayed retained functional activity (FIG. 7). These results indicate that our polymeric bilayers protect protein integrity during lyophilization and extended dehydrated storage.

TABLE 2
Protein rates and percent increase in fluorescence intensity
of pGUP controls (non-lyophilized) and after lyophilization
(24 h and 120 h). +Ag indicates added agonist; Ctl samples represent
basal activity (without agonist). Percent intensity is indicative
of active 5-HT_{1A}R population.

| | | Rate | Std Error | | |
|---|---|---|---|---|---|
| Lyophilization | | (×10⁻² | (×10⁻² | % | Std |
| (h) | Sample | min⁻¹) | min⁻¹) | Intensity | Error |
| Non lyophilized | Ctl | 2.4 | 0.1 | 85% | 11% |
| 24 | Ctl | 3.0 | 0.1 | 73% | 8% |
| 120 | Ctl | 2.5 | 0.2 | 83% | 12% |
| Non lyophilized | +Ag | 7.7 | 0.1 | 80% | 10% |
| 24 | +Ag | 7.3 | 0.2 | 77% | 7% |
| 120 | +Ag | 6.5 | 0.1 | 85% | 9% |

TABLE 3
Rates of pGUPs without bound spiperone. pGUP curves from FIG.
6 were fitted to a mechanistic growth single exponential fit
to determine the quantitative rates of the agonist and control
samples at different lyophilization time points. The 0 h, non-
lyophilized agonist induced and control samples display a faster
rate than the 24 h and 120 h lyophilized samples. Nonetheless
protein retains agonist-induced activity and protein functionality
suggesting that the polymer stabilizes the GPCR.

| | | Rate | Std Error |
|---|---|---|---|
| Lyophilization | | (×10⁻² | (×10⁻² |
| (h) | Sample | min⁻¹) | min⁻¹) |
| 0 | Ctl | 5.7 | 0.1 |
| 24 | Ctl | 3.0 | 0.1 |
| 120 | Ctl | 3.6 | 0.1 |
| 0 | +Ag | 16.0 | 0.4 |
| 24 | +Ag | 5.3 | 0.1 |
| 120 | +Ag | 6.8 | 0.2 |

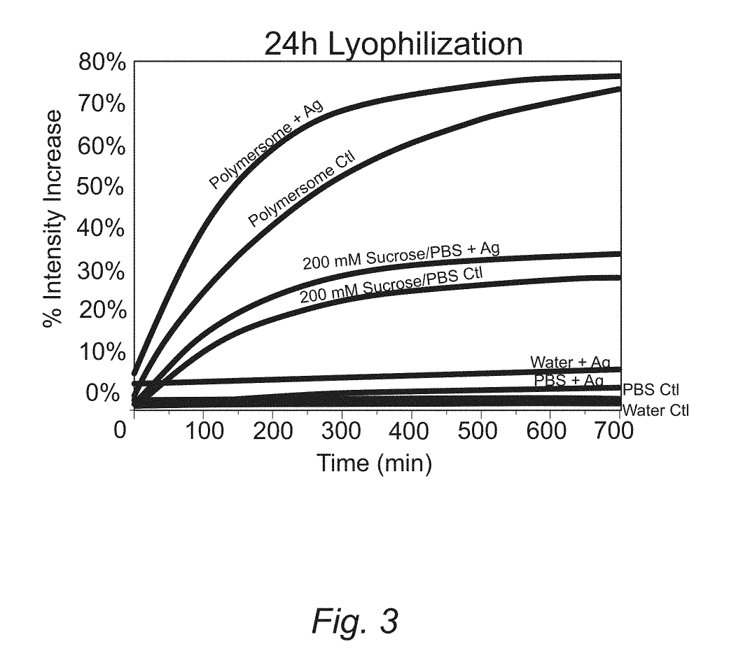

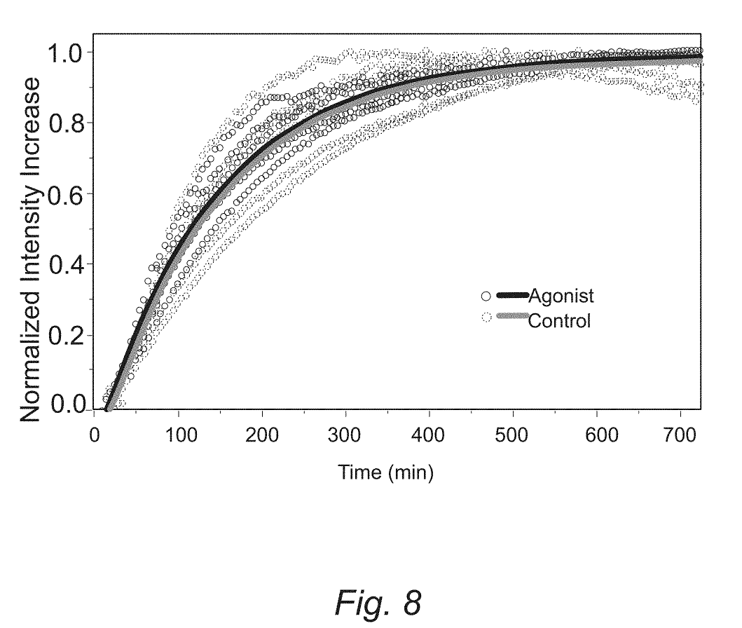

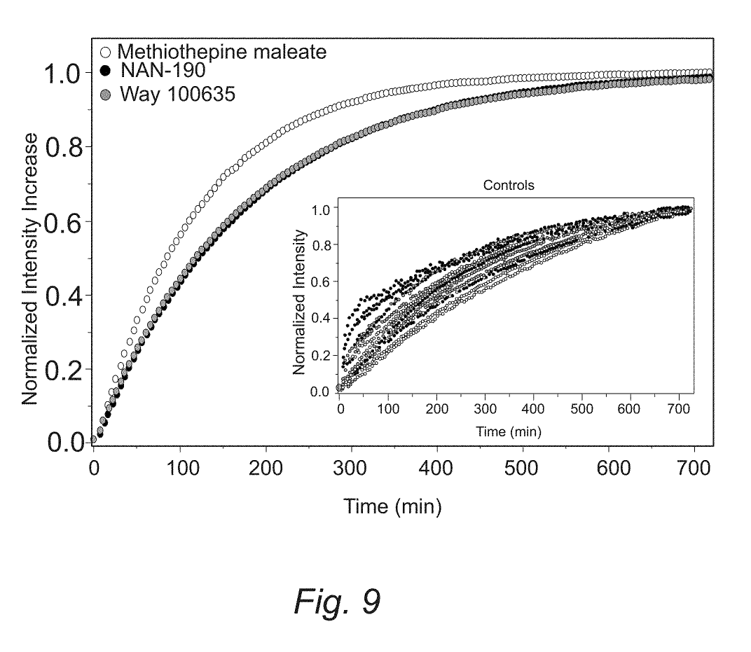

Proteins may be stabilized by ligands and sugars, which can aid in keeping their functional integrity during lyophilization.²⁴⁻²⁵ To determine if the stabilization in our systems is due in part from concentrated sugar present in the buffer used for pGUP formation (200 mM sucrose in PBS), membrane fragments of 5-HT_{1A}R were bound to spiperone and then diluted in deionized water, PBS (pH 7.4), or 200 mM sucrose in PBS (pH 7.4) with concentrations similar to our pGUP system. After 24 hours of lyophilization, samples were rehydrated and assessed for protein function. 5-HT_{1A}R membrane fragments diluted in Milli Q water or PBS did not retain its function (FIG. 3). In 200 mM sucrose in PBS, the protein displays a similar functional rate for both control and agonist treated samples. Despite displaying protein function, the rates do not discriminate between control and agonist exposed samples suggesting that the protein has lost its agonist binding ability (FIG. 8). Furthermore, the percent intensity increase of these samples were well below that of the pGUP samples, indicating that only a small population of proteins retained some function (FIG. 3). Thus, while these results suggest that sugar stabilizes 5-HT_{1A}R to some extent during lyophilization, its protective ability is much lower than the overall stability and protection offered by our pGUPs, which not only retain vesicle shape and size, but also retain protein functional integrity.

Using an agarose rehydration technique, we not only show successful incorporation of GPCR 5-HT_{1A}R into polymeric vesicles in the form of pGUPs but also show increased protein stability during lyophilization and extended dehydrated storage. Successfully reconstituted 5-HT_{1A}R in diblock copolymer pGUP vesicles on the micrometer scale exhibits expected physiological responses to different antagonists and at a variety of concentrations. Rehydration of pGUPs after 24 hours and 120 hours of lyophilization retains vesicle size and consistent protein function and offers increased stability as compared to buffered sugar solutions. Thus, we offer a simple platform to investigate protein function in polymer vesicles in the form of pGUPs. Extension of this work to other types of GPCRs is currently being conducted.

Materials

Diblock copolymer poly(butadiene-b-ethyleneoxide) (PBd(650)-PEO(400)) was acquired from Polymer Source (Canada) and used without further purification. ATTO-488-DPPE fluorescence tag was used as indicated (ATTO-TEC, Germany). All reagents such as but not limited to low melt-temperature agarose, phosphate buffered saline (PBS), dimethyl sulfoxide (DMSO), chloroform (CHCl₃), methanol (MeOH), sucrose, glucose, WAY 100635, methiothepin maleate and agonist 8-Hydroxy-2-(dipropylamino)tetralin hydrobromide (8-OH-DPAT) were of analytical grade from Sigma Aldrich, USA. Membrane fragments containing 5-HT_{1A}R (Perkin Elmer, USA), 5-HT_{1A}R monoclonal (Millipore, USA) and G_{αi} protein antibodies (Thermo Fisher, USA), antagonist spiperone (Tocris, UK) and NAN-190 (Tocris, UK) were used without further purification. Sykes-Moore chambers (Bellco, USA), standard 25 mm no. 1 glass coverslips (ChemGlass, USA), and flat bottom 96-well plates (BD Biosciences, USA) were used throughout all experiments. 18.2 MΩ-cm Milli-Q water was used in all experiments (EMD Millipore, USA). Protein desalting micro spin columns (Thermo Scientific, USA) and NHS-rhodamine (Thermo Scientific, USA) were used as per the manufacturer's instructions. BODIPY-GTPγS and QSY7 were obtained from Life Technologies, USA and used as directed by manufacturer's instructions. Solutions of ligands were made to 10 mM in DMSO. 8-OH-DPAT was made at 10 mM in water.

Fabrication of Polymer Vesicles and Protein Incorporation

25 mm no. 1 coverslips were cleaned via sonication in MeOH for 30 minutes at 35° C. Coverslips were dried and were further plasma treated in a PDC-32G benchtop plasma cleaner (Harrick Plasma, USA) for 15 minutes. Coverslips were held in Sykes-Moore chambers for vesicle formation. Protein-incorporated polymeric giant unilamellar protein-vesicles (pGUPs) were formed using methods similar to those previously published for lipid vesicles. Briefly, a 1:1 v/v mixture of membrane fragment suspension and agarose (3% w/v) at 45° C. was drop casted onto coverslips for thin film formation. The hydrogel film was allowed to gel and a film of polymer solution (5 mg/ml in CHCl₃) was subsequently made. Solvent was evaporated using a stream of N₂ gas. The films were hydrated with 200 mM sucrose in PBS (pH 7.4) with 100 uM spiperone and BODIPY-γ-FL-GTP (70 nM final concentration) for 20 minutes. Vesicles were harvested from the coverslip and diluted in 3× of an isoosmotic glucose solution (200 mM glucose in PBS, pH 7.4). (p)GUPs were allowed to sediment in glucose for 30 minutes at room temperature.

5-HT_{1A} Activity Assay via Microplate Reader

Prepared and settled pGUPs were transferred to a flat bottom 96-well plate. Agonist 8-OH-DPAT was added to each well to a final concentration of 150 nM immediately prior to reading fluorescence. Control pGUPs were run without the addition of the agonist. Rehydration buffer with and without agonist and protein were read as positive and negative controls. Samples were read at physiological temperature (37° C.) every 5 minutes for twelve hours to ensure complete activity assessment. Fluorescence reading of BODIPY-GTPγS unquenching was done on a Biotek Synergy H4 Microplate Reader equipped with a xenon flash lamp. Excitation was set to 485/20 nm and emission at 528/20 nm at a read height of 7 mm.

pGUP Lyophilization

Protein incorporated vesicles were prepared and were not subjected to sedimentation. Samples were transferred to microcentrifuge tubes with a hole pierced through the top to allow for water evaporation during lyophilization. Samples were first flash-frozen in liquid nitrogen for 5 minutes. Immediately following, samples were placed under a vacuum at 0.5 torr overnight to complete lyophilization. If needed, samples were stored with dessicant at −20° C.

Antibody Labeling

5-HT_{1A}R monoclonal and G_{αi} antibodies were equilibrated to room temperature and conjugated to NHS rhodamine in DMSO at 10× molar excess. Sodium bicarbonate was added as per manufacturer's instructions to raise the solution pH to 8.0. The solution was allowed to react for one hour at room temperature and then overnight at 5° C. Rhodamine-labeled antibodies were subsequently desalted using spin columns according the manufacturer's instructions. Labeled antibody UV-vis absorbance was read on a NanoDrop ND-1000 (Thermo Fisher, USA). Rhodamine-labeled Gal antibody concentration was determined to be 22 uM and labeling efficiency was calculated to be 1.15. Rhodamine labeled 5-HT_{1A}R monoclonal antibody concentration was 3.2 uM with 1.41 labelling efficiency.

5-HT_{1A} Receptor and G Protein Identification via Antibody Binding

An aliquot of 5-HT_{1A}R membrane fragment was incubated with 1:1000 labeled antibody, either 5-HT_{1A}R monoclonal or G_{αi} at 37° C. for 1 hour. Labeled protein was used in the agarose film for pGUP formation. pGUPs were formed as previously described with spiperone and BODIPY-GTPγS omitted from the rehydration buffer. Polymer solutions included 0.2% ATTO-4880-DPPE for ease of imaging. pGUPs were harvested, settled in an isoosmotic glucose solution and transferred to observation chambers. pGUPs imaged at 491 nm and 561 nm excitation corresponding to 523 nm and 575 nm emission respectively.

Antibody quenching to determine protein orientation

5-HT_{1A}R membrane fragments were incubated with rhodamine-labeled 5-HT_{1A}R monoclonal antibodies or rhodamine-labeled G_{od} monoclonal antibodies, 1:1000 dilution. The labeled protein mixture was used in the agarose film for pGUP formation. For control vesicles, labeled protein was omitted from the preparation and instead 0.2% ATTO-488-DPPE was used. pGUPs were harvested, settled, and placed in observation chambers. pGUPs were imaged via epifluorescent microscopy prior to quenching. QSY7, a membrane impermeable quenching molecule, was then added to the observation chambers, 1:1000 dilution, and incubated in the dark for 10 minutes. pGUPs were imaged before and after incubation and the amount of quenched intensity was analyzed. Control data without tagged protein shows 50% quenching of ATTO-488-DPPE fluorophore, indicative of even distribution of fluorescent dye in the vesicles (FIG. 4).

Microscopy

Imaging was done on a TI-Eclipse inverted microscope (Nikon, Japan) equipped with a spinning-disc CSUX confocal head (Yokogawa, Japan) and a 16-bit Cascade II 512 EMCCD camera (Photometrics, USA). Confocal excitation of fluorophores was done using 50 mW solid-state lasers at 491 nm for ATTO-488-DPPE and 561 nm for rhodamine (Coherent Inc., Germany). All confocal images were taken using a Plan-Apo 60× NA1.43 oil immersion Nikon objective. Epifluorescence imaging was performed on the same microscope with illumination from a 130 W mercury lamp (Intensilight, Nikon, Japan). Rhodamine emission was excited using a green filter (528-552 nm bandpass, 540 nm cut-on wavelength). Temperature control during imaging was performed using a heating-cooling stage with a stability and accuracy of 0.1° C. (Bioscience Tools, USA).

Differential interference contrast (DIC) images were collected on an Axio Observer Z1 (Zeiss, Germany) inverted microscope using an EC Plan-Neofluar 40× objective and equipped with a Hamamatsu CMOS camera (Hamamatsu, Japan). Illumination was provided by a halogen lamp 12V 100W using a differential interference contrast prism with polarizer (Zeiss, Germany).

Image Processing

All images were processed and analyzed using ImageJ. Particle analysis and measurements were performed using ImageJ Analyze Tools. Fluorescent micrographs of vesicles using 491 nm excitation are shown using the ImageJ green lookup table and micrographs using 561 nm excitation are shown using the Image J orange hot lookup table. All images are presented without any further processing adjustments or corrections and are scaled from minimum to maximum intensity.

Data Analysis

Fluorescence microtiter results were collect and analyzed using JMP. Six separate observations were fitted and averaged to obtain a single curve with standard error mean values. Rates were fitted to the JMP Mechanistic Growth curve using a single exponential. Statistical analysis using a Tukey-Kramer pairwise comparison of means were done using JMP with a 95% confidence interval (α=0.05).

As required, detailed embodiments of the present invention are disclosed herein; however, it is to be understood that the disclosed embodiments are merely exemplary of the invention that may be embodied in various and alternative forms. The figures are not necessarily to scale; some features may be exaggerated or minimized to show details of particular components. Therefore, specific structural and functional details disclosed herein are not to be interpreted as limiting, but merely as a representative basis for teaching one skilled in the art to variously employ the present invention.

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

### REFERENCES

1. Discher, B. M., Science 1999, 284 (5417), 1143-1146.
2. Discher, D. E.; Eisenberg, A., Science 2002, 297 (5583), 967-973.
3. Rodríguez-García, R.; Mell, M.; López-Montero, I.; Netzel, J.; Hellweg, T.; Monroy, F., Soft matter 2011, 7 (4), 1532-1542.
4. Jain, J. P.; Ayen, W. Y.; Kumar, N., Curr. Pharm. Des. 2011, 17, 65-79.
5. Le Meins, J. F.; Schatz, C.; Lecommandoux, S.; Sandre, O., Mater. Today 2013, 16 (10), 397-402.
6. Peters, R. J.; Marguet, M.; Marais, S.; Fraaije, M. W.; van Hest, J. C.; Lecommandoux, S., Angew. Chem. Int. Ed. 2014, 53 (1), 146-150.
7. Kumar, M.; Grzelakowski, M.; Zilles, J.; Clark, M.; Meier, W., Proc. Natl. Acad. Sci. U.S.A 2007, 104 (52), 20719-24.
8. Amado, E.; Schöps, R.; Brandt, W.; Kressler, J., ACS Macro Lett 2012, 1 (8), 1016-1019.
9. Katritch, V.; Cherezov, V.; Stevens, R. C., Annu. Rev. Pharmacol. Toxicol. 2013, 53, 531-556.
10. Drews, J., Science 2000, 287, 1960-1964.
11. Lefkowitz, R. J., Trends Pharmacol. Sci. 2004, 25 (8), 413-422.
12. May, S.; Andreasson-Ochsner, M.; Fu, Z.; Low, Y. X.; Tan, D.; de Hoog, H. P.; Ritz, S.; Nallani, M.; Sinner, E. K., Angew. Chem. Int. Ed. 2013, 52 (2), 749-753.
13. de Hoog, H. P.; Lin JieRong, E. M.; Banerjee, S.; Decaillot, F. M.; Nallani, M., PLOS One 2014, 9 (10), e110847.
14. Gutierrez, M. G.; Malmstadt, N., J. Am. Chem. Soc. 2014, 136 (39), 13530-13533.
15. Hansen, J. S.; Thompson, J. R.; Helix-Nielsen, C.; Malmstadt, N., J. Am. Chem. Soc. 2013, 135 (46), 17294-17297.
16. Bermudez, H.; Brannan, A. K.; Hammer, D. A.; Bates, F. S.; Discher, D. E., Macromolecules 2002, 35, 8203-8208.
17. Horger, K. S.; Estes, D. J.; Capone, R.; Mayer, M., J. Am. Chem. Soc. 2009, 131, 1810-1819.
18. Bordignon, E., Top. Curr. Chem. 2012, 321, 121-157.
19. Newman-tancredi, A.; Cussac, D.; Marini, L.; Millian, M. J., Mol. Pharmacol. 2002, 62, 590-601.
20. Bojarski, A. J.; Mokrosz, M. J.; Duszyńska, B.; Koziol, A.; Bugno, R., Molecules 2004, 9, 170-177.
21. Newman-Tancredi, A.; Verrièle , L.; Chaput, C.; Millan, M. J., Naunyn Schmiedebergs Arch. Pharmacol. 1998, 357, 205-217.
22. Chen, C.; Han, D.; Cai, C.; Tang, X., J. Control. Release 2010, 142 (3), 299-311.
23. MacDonald, R. C.; Jones, F. D.; Qiu, R., Biochim. Biophys. 1994, 1191, 362-370.
24. Leslie, S. B.; Israeli, E.; Lighthart, B.; Crowe, J. H.; Crowe, L., M., Appl. Environ. Microbiol. 1995, 61 (10), 3592-3597.
25. Wolkers, W. F.; Oldenhof, H.; Tablin, F.; Crowe, J. H., Biochim. Biophys. Acta 2004, 1661 (2), 125-34.

What is claimed is:

1. A composition comprising:
a unilamellar polymersome having an exterior surface and an interior surface, the interior surface defining a central cavity, the unilamellar polymersome including a polymeric layer and at least one functional G protein-coupled receptor incorporated into the polymeric layer.

2. The composition of claim 1 wherein the polymeric layer is a polymeric bilayer.

3. The composition of claim 1 wherein the polymer layer includes a block copolymer.

4. The composition of claim 3 wherein the block copolymer has a number average molecular weight from about 2,000 to about 100,000 Daltons.

5. The composition of claim 3 wherein the block copolymer has a hydrophilic mass fraction from about 20% to about 45% by weight of the block copolymer.

6. The composition of claim 3 wherein the polymer layer includes a hydrophilic block selected from the group consisting of poly(ethylene glycol), poly(2-methyloxazoline), poly(acrylic acid), poly(ethylene oxide), poly(methacrylic acid), poly(2-acrylamido, 2-methyl propane sulfonic acid), poly(acrylamide), poly(2-dimethylaminoethyl methacrylate), and combinations thereof.

7. The composition of claim 3 wherein the polymer layer includes a hydrophobic block selected from the group consisting of polydimethylsiloxane, poly(caprolactone), poly(lactide), poly(methyl methacrylate), poly(propylene), poly(butadiene), poly(styrene), poly(isoprene), poly(ethylene), poly(ethylene propylene), poly(ethylene butene) and combinations thereof.

8. The composition of claim 3 wherein the polymer layer includes a hydrophilic block selected from the group consisting of poly(ethyleneoxide).

9. The composition of claim 3 wherein the polymer layer includes a hydrophilic block selected from the group consisting of poly(butadiene).

10. The composition of claim 1 wherein the G protein-coupled receptor includes a receptor selected from the group consisting of rhodopsin-like receptors, receptors of the secretin receptor family, metabotropic glutamate/pheromone receptors, fungal mating pheromone receptors, cyclic AMP receptors, and frizzled/smoothened receptors.

11. The composition of claim 1 wherein the G protein-coupled receptor includes a receptor selected from the group consisting of angiotensin receptors, bombesin receptors, bradykinin receptors, calcitonin, parathyroid hormone, secretin receptors, chemokine receptors, chemotactic peptide receptors, c5a receptor, cholecystokinin/gastrin receptors, corticotropin (ACTH) receptor, endothelin receptors, glycoprotein hormones receptors, melanocortin receptors, motilin receptors, neuropeptide Y receptors, neurotensin receptors, oploid receptors, mu opioid receptor, delta opioid receptor, kappa opioid receptor, nociceptin, opioid receptor, releasing hormone receptors, somatostatin receptors, tachykinin receptors, thrombin/protease receptors, vasopressin/oxytocin receptors, acetylcholine (muscarinic) receptors, adrenergic receptors, dopamine receptors, histamine receptors, serotonin receptors, adenosine and other adenine nucleotide receptors, cannabinoids receptors, prostanoids and paf receptors, calcium receptors, opsins, viral receptors, and orphan receptors.

12. The composition of claim 1 wherein the G protein-coupled receptor is 5-HT_{1A}R.

13. The composition of claim 1 further comprising a payload compound disposed within the central cavity.

14. A method for forming the composition of claim 1, the method comprising:
forming a film of a protein and polymer on a substrate; and
rehydrated the film to form the polymersome.

15. The method of claim 14 wherein the film further includes a payload molecule.
